(19) United States
(12) Patent Application Publication
(10) Pub. No.: US 2022/0411524 A1
(43) Pub. Date: Dec. 29, 2022

(54) COMBINATION IMMUNOTHERAPY OF IL-15 AND CD40 AGONIST IN CANCER TREATMENT

(71) Applicants: UNIVERSITEIT ANTWERPEN, Antwerpen (BE); UNIVERSITAIR ZIEKENHUIS ANTWERPEN, Edegem (BE)

(72) Inventors: Evelien SMITS, Antwerpen (BE); Jonas VANAUDENAERDE, Borgerhout (BE); Marc PEETERS, Waasmunster (BE)

(73) Assignees: UNIVERSITEIT ANTWERPEN, Antwerpen (BE); UNIVERSITAIR ZIEKENHUIS ANTWERPEN, Edegem (BE)

(21) Appl. No.: 17/781,583

(22) PCT Filed: Dec. 4, 2020
(86) PCT No.: PCT/EP2020/084665

§ 371 Date: Jun. 1, 2022

(30) Foreign Application Priority Data
Dec. 6, 2019 (EP) 19214049.9

(51) Int. Cl. C07K 16/28; A61K 38/20

(52) U.S. Cl. CPC C07K 16/2878; A61K 38/2086; A61K 2039/505

(57) ABSTRACT

The present invention relates to the field of combination immunotherapy, more in particular to a combination comprising IL-15 and a CD40 agonist for use in the treatment of cancer, such as and not limited to, pancreatic cancer (e.g. pancreatic ductal adenocarcinoma and pancreatic neuro-endocrine tumours).

### FIELD OF THE INVENTION

The present invention relates to the field of combination immunotherapy, more in particular to a combination comprising IL-15 and a CD40 agonist for use in the treatment of cancer, such as and not limited to, pancreatic cancer (e.g. pancreatic ductal adenocarcinoma and pancreatic neuro-endocrine tumours).

### BACKGROUND TO THE INVENTION

Pancreatic ductal adenocarcinoma (PDAC) is the third most lethal cancer worldwide with a 5-year survival of barely 8%. It even is projected to become the second leading cause of cancer-related death by 2030. To date, it remains one of the most aggressive and challenging gastrointestinal malignancies due to a complex tumour microenvironment including a strong desmoplastic reaction, a low immunogenicity and finally a molecular signature in favour of the tumour, driven by loss of multiple tumour suppressor genes.

To date, no improvement in survival has been achieved, rendering PDAC a disease which represents the very definition of an urgent unmet need for novel therapeutic approaches to finally improve the outcome of patients. About 85% of the patients is not eligible for curative surgical resection due to locally advanced or metastatic disease at the time of diagnosis. Hence, they are treated with either FOLFIRINOX or Gemcitabine/nab-Paclitaxel depending on their physical fitness since the first has major toxicity issues but the latter only limited impact. New promising approaches, successful in other cancer types, unfortunately failed to reach improvement over gemcitabine, including anti-PD-1 and anti-CTLA-4.

Agonistic CD40 antibodies have shown promising results in mice bearing spontaneous pancreatic KPC tumours. This led to clinical trials where several patients were treated with gemcitabine and a CD40 agonist with modest results in most patients. However, there remains a big margin to increase the potential of CD40 agonists, possible by combinations with other compounds.

We have shown that IL-15 has the potential to kill not only PDAC tumour cells but also the stromal pancreatic stellate cells (PSC) which are responsible for the poor response to treatment. IL-15 is a versatile cytokine which stimulates both T cell proliferation and generation of cytotoxic lymphocytes, as well as activation and expansion of natural killer (NK) cells. Furthermore, it has the capability to induce CD8 memory cells, thereby playing a crucial role in maintaining long-lasting immune responses to malignant cells and possible prevention of relapse. All these features render it a highly attractive cancer immunotherapeutic as confirmed by its high rank in the NCI's top 20 immunotherapeutic drugs with the greatest potential for broad usage in cancer therapy.

While both molecules have previously been shown to have potential in the treatment of pancreatic tumours, we have now surprisingly found that in combination they exhibit additive effects in terms of enhanced anti-tumour effect resulting in profound survival increase and even complete cure of the majority of tumours. Also, a striking dose reduction of CD40 agonist was possible by adding IL-15, thereby also reducing the risk of side-effects. Hence, we found that by combining both, at least one of the components can be used at subtherapeutic doses, yet still obtaining a similar efficacy.

### SUMMARY OF THE INVENTION

The present application is directed to a combination comprising IL-15 and a CD40 agonist for use in the treatment of a pancreatic cancer. In particular, the present invention provides a combination of IL-15 and CD40 agonist for use in the treatment of a pancreatic cancer, wherein at least one of said IL-15 and CD40 agonist are used in a subtherapeutic dose.

In a particular embodiment, said CD40 agonist is administered/used at a dose of from about 20 to about 800 μg per kg body weight, preferably from about 30 to about 600 μg per kg body weight, most preferably from about 40 to about 300 μg per kg body weight.

In another particular embodiment, said IL-15 is administered at a dose of from 0.1 to about 50 μg per kg body weight, preferably from about 0.1 to 20 μg per kg body weight, most preferably from about 0.1 to about 2 μg per kg body weight.

In a further embodiment, said IL-15 is administered intravenously at a dose of less than 0.3 μg per kg body weight. In particular where IL-15 is administered via a bolus IV injection, the dose may be less than 0.3 μg per kg body weight. Alternatively, where IL15 is administered via a continuous IV drip system, the dose may be about or below 2 μg per kg body weight. Alternatively, said IL-15 is administered intradermally or subcutaneously at a dose of less than 2 μg per kg body weight.

In yet a further embodiment, the present invention provides a combination as defined herein, wherein at least one of the following applies:
said CD40 agonist is used at a dose of less than 300 μg per kg body weight;
said IL-15 is administered intravenously at a dose of less than 0.3 μg per kg body weight;
said IL-15 is administered via a bolus injection intravenously at a dose of less than 0.3 μg per kg body weight;
said IL-15 is administered via a continuous drip system intravenously at a dose of about or below 2 μg per kg body weight; or
said IL-15 is administered subcutaneously or intradermally at as dose of less than 2 μg per kg body weight.

In another particular embodiment, the combination is in the form of a pharmaceutical composition.

In another particular embodiment, said CD40 agonist is a CD40 antibody or antigen binding fragment thereof such as selected from: Selicrelumab, APX005M, ChiLob7/4, ADC-1013, SEA-CD40, CDX-1140.

In another particular embodiment, said CD40 agonist is selected from: CD40L, trimers of CD40L, HERA-CD40L.

In another particular embodiment, said IL-15 and said CD40 agonist are administered simultaneously.

In a further embodiment, said IL-15 and said CD40 agonist are administered intravenously or subcutaneously.

In yet a further embodiment, the pancreatic cancer is selected from: pancreatic ductal adenocarcinoma, pancreatic neuroendocrine tumours.

In particular, the inventors found that said combination comprising IL-15 and a CD40 agonist exhibits enhanced anti-tumour effect resulting in profound survival increase and even complete cure of the majority of tumours. Moreover, the inventors have surprisingly found that a striking dose reduction of CD40 agonist was possible by adding IL-15.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Tumour Kinetics and Survival.

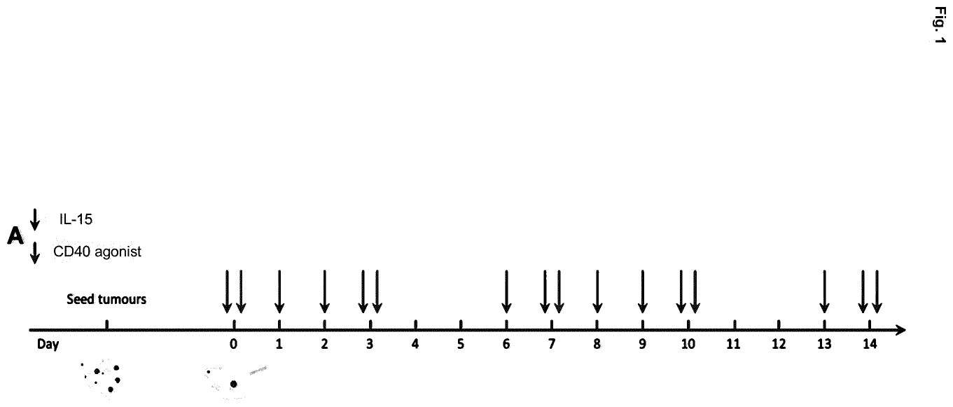

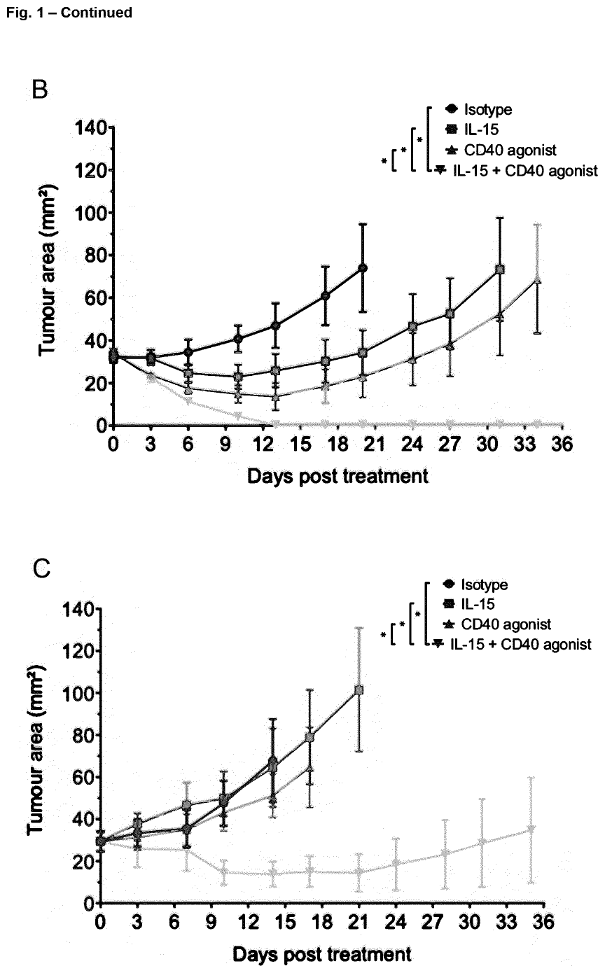

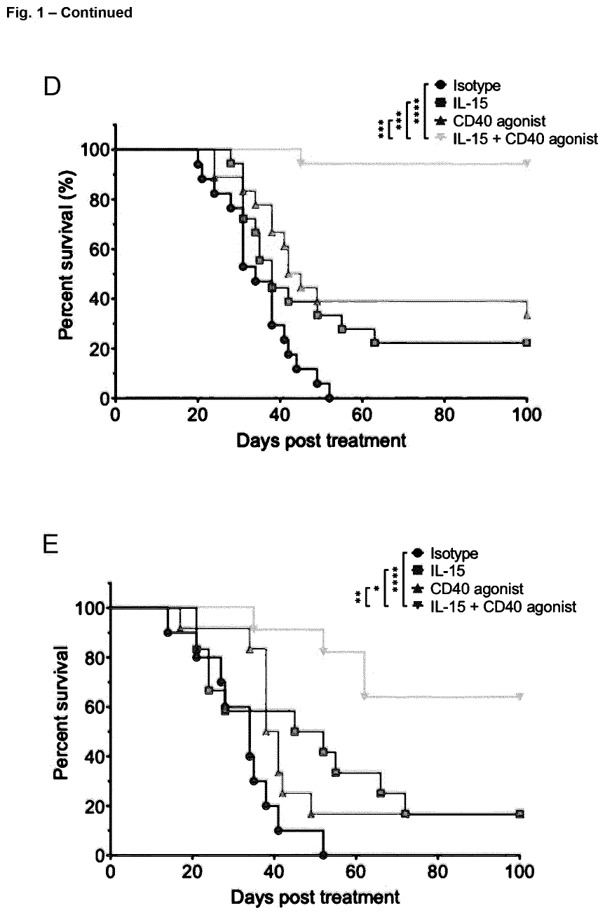

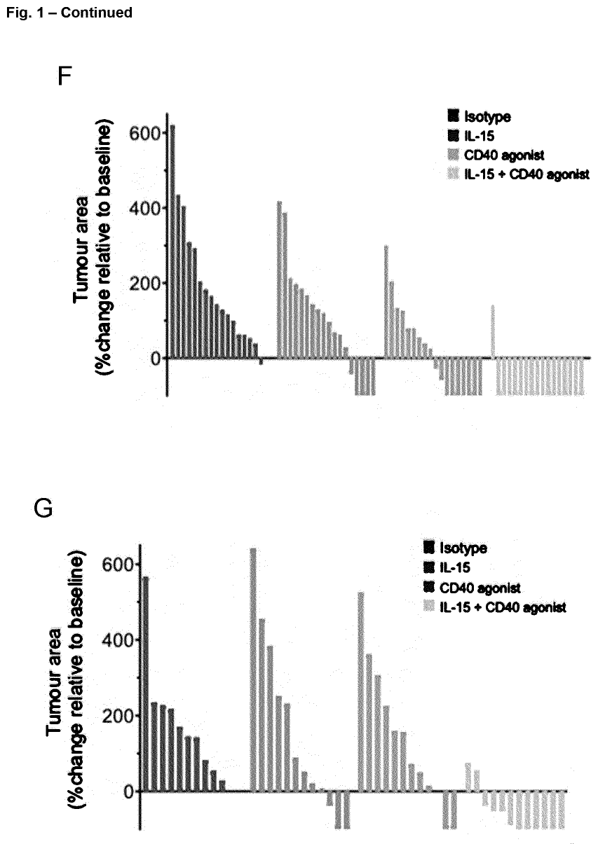

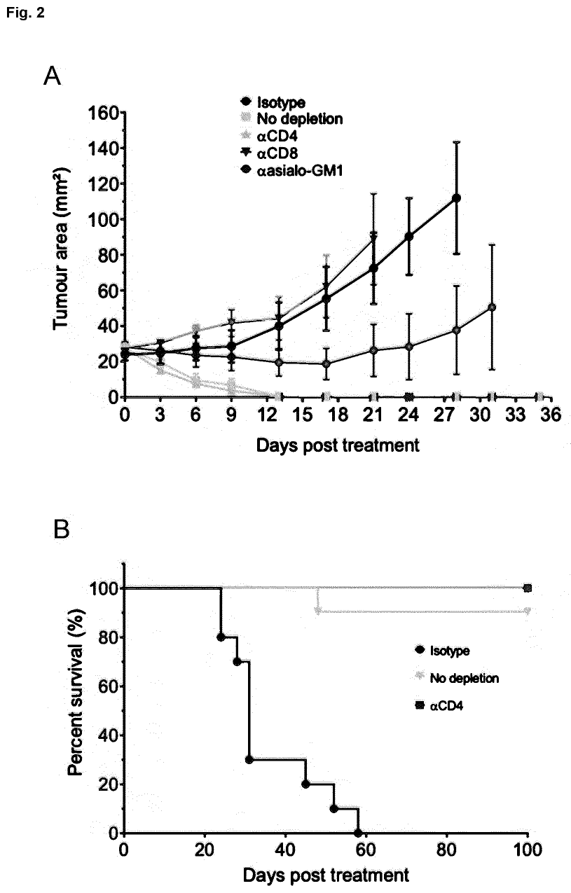

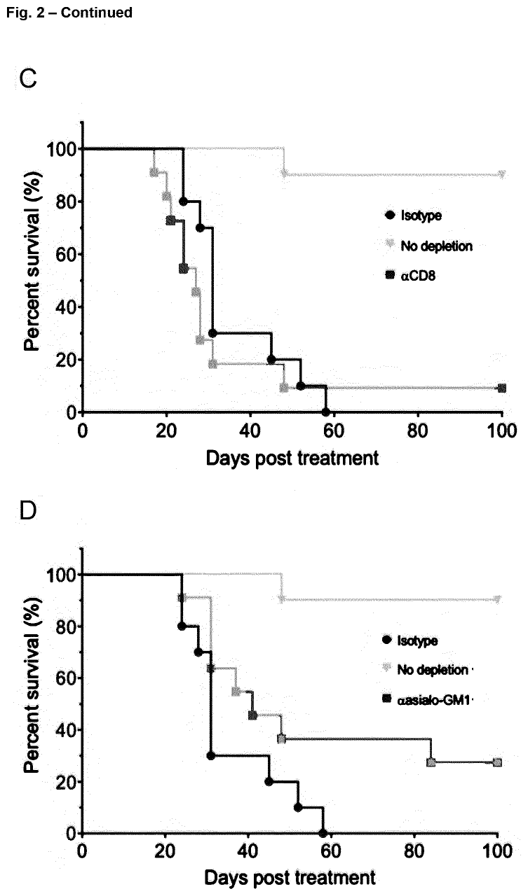

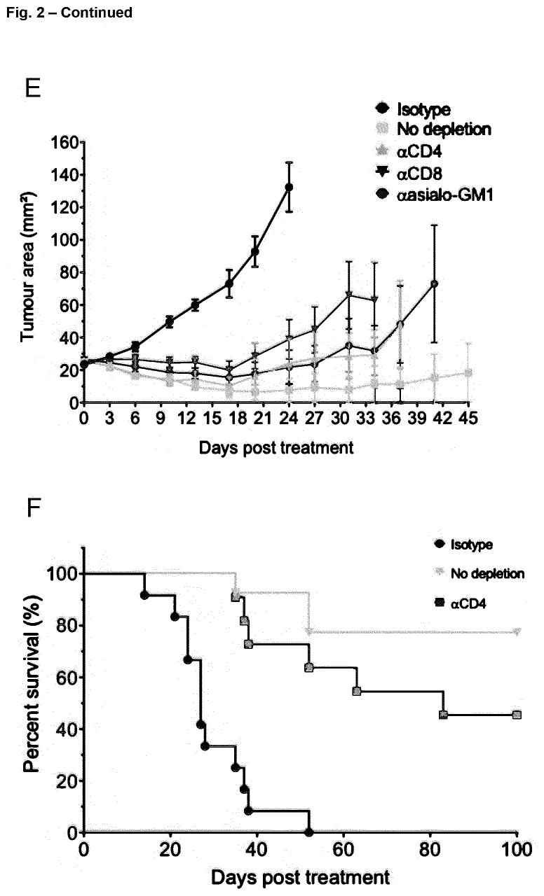

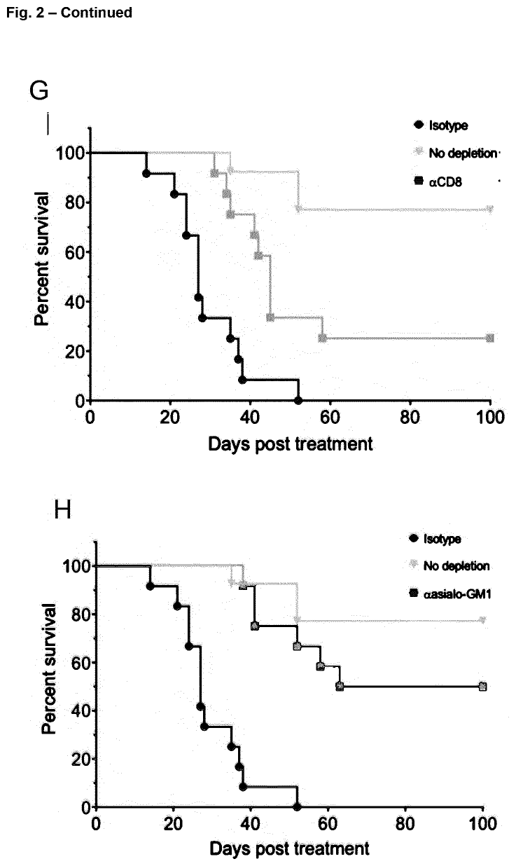

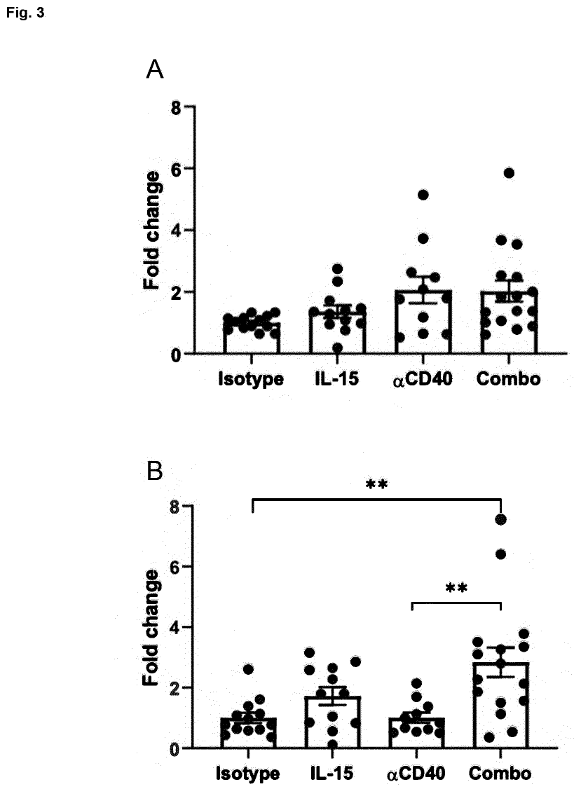

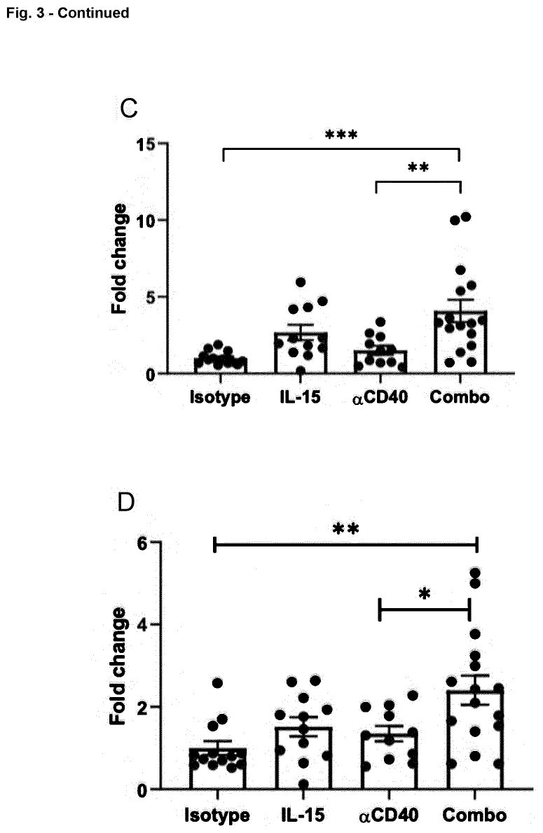

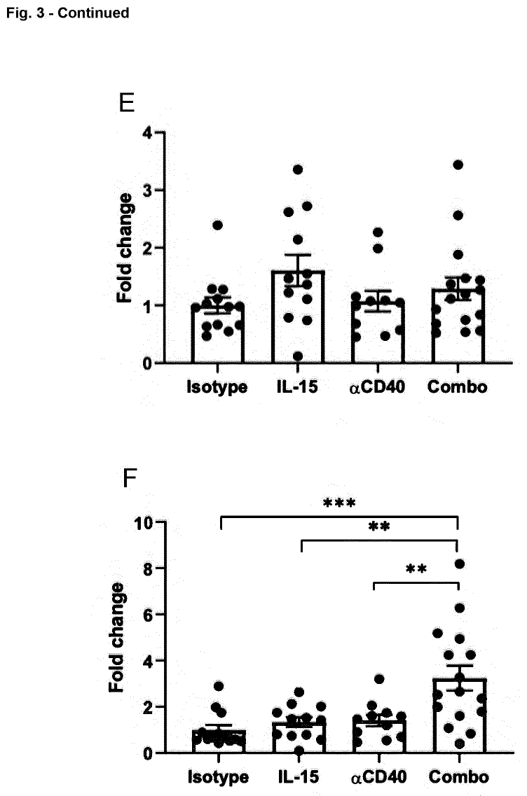

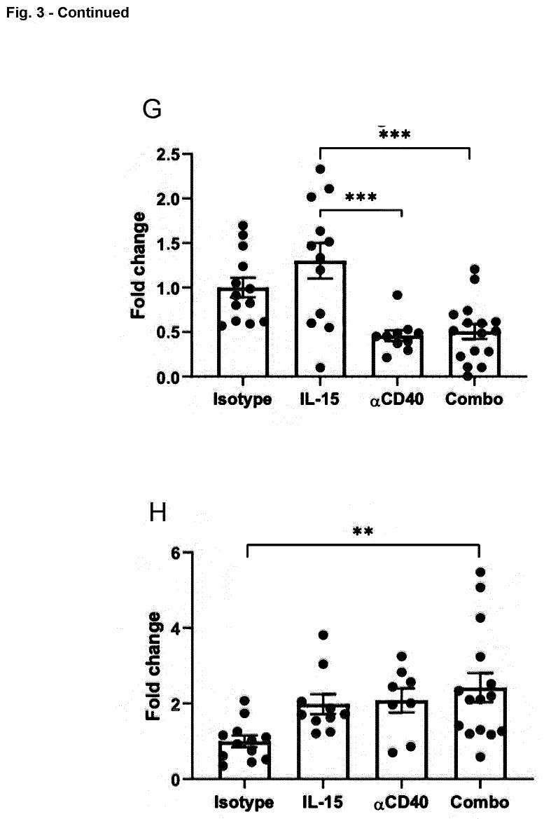

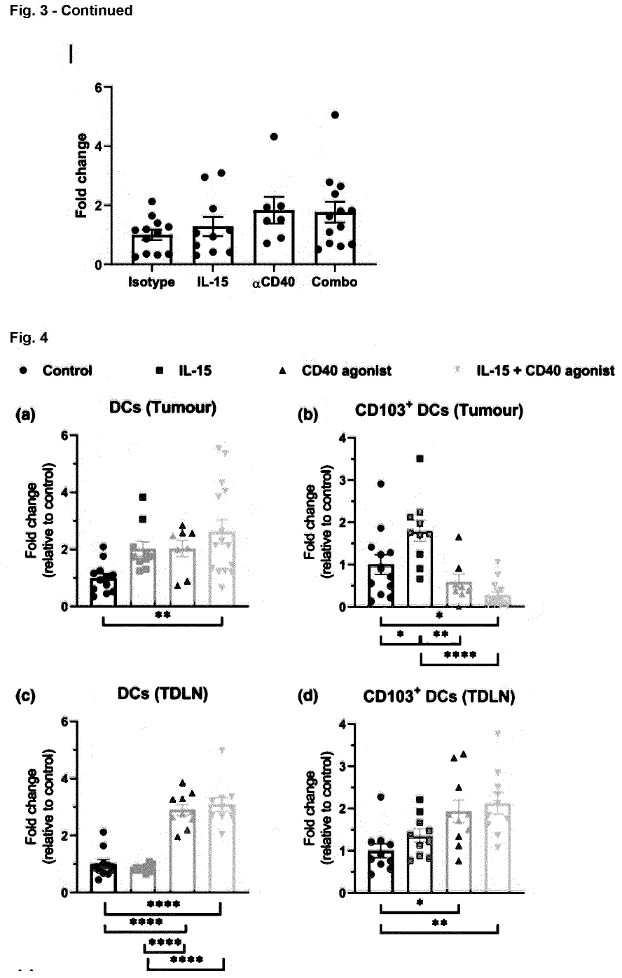

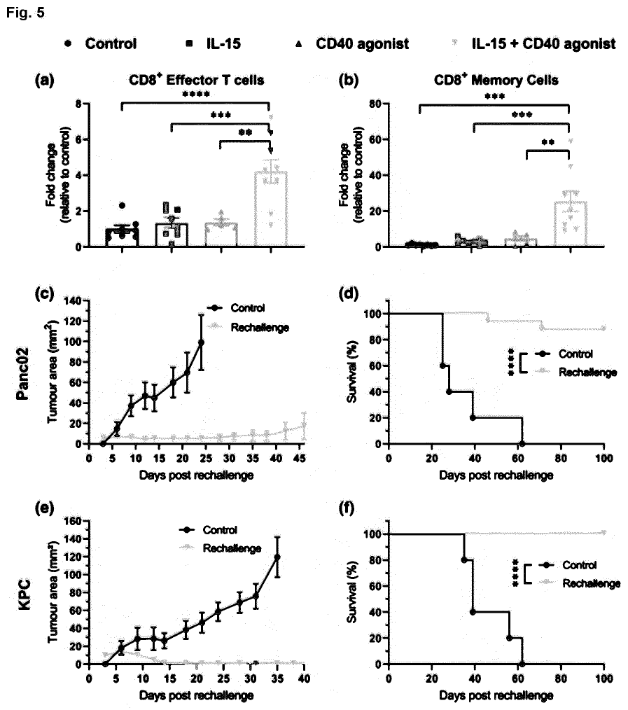

C57BI/6j mice were injected with either 0.5×10⁶ Panc02 cells (see FIG. 1B, 1D, 1F) or KPC (see FIG. 1C, 1E, 1G) cells subcutaneously. When tumours reached a size of 25-35 mm², mice were randomised and treated with isotype control, IL-15, CD40 agonist or IL-15+CD40 agonist according the treatment scheme (FIG. 1A). Timing of dosing is indicated for IL-15 (2.5 μg) with black arrows and for CD40 agonist or the corresponding isotype with red arrows (12.5 μg for Panc02 and 200 μg or 100 μg for KPC). In FIG. 1B, 1C tumour kinetics are depicted (n=6 mice per group, representative data of 3 (Panc02) or 2 (KPC) independent experiments. Two-way ANOVA with Bonferroni posthoc. In FIG. 1D, 1E, the survival of Panc02 and KPC mice treated as indicated is illustrated. Pooled data of 3 (Panc02) or 2 (KPC) independent experiments. Survival was determined by tumour size reaching 150 mm². Mantel-Cox test. In FIG. 1F, 1G waterfall plots showing the % fold change relative to baseline after 34 days (Panc02) or 35 days (KPC) are illustrated. All data represent mean±SEM. ns p≥0.05;*p<0.05;**p≤0.01;***p≤0.001;****p≤0.0001.

FIG. 2: Immune Cell Depletion

C57BI/6j mice bearing Panc02 tumours (see FIG. 2A, 2B, 2C, 2D) or KPC tumours (see FIG. 2E, 2F, 2G, 2H) were treated with isotype control or the IL-15+CD40 agonist combination regimen alone or with depleting antibodies against CD4, CD8, asialo-GM1 (NK cell depletion). FIG. 2A, 2E illustrate tumour kinetics of Panc02 or KPC tumours either non-treated (isotype), treated with the combination regimen only (no depletion) or combination and depletion antibodies. Two-way ANOVA with Bonferroni posthoc. Data points represent mean±SEM. n=5-7 mice/group. FIG. 2B to FIG. 2D, and FIG. 2F to FIG. 2H illustrate the survival of Panc02 (FIG. 2B-2D) or KPC (FIG. 2F-2H) bearing mice either non-treated (isotype), treated with the combination regiment (no depletion) or the combination and depletion antibodies against CD4 (FIG. 2B, 2F), CD8 (FIG. 2C, 2G), asialo-GM1 (FIG. 2D, 2H). Data pooled from 2 independent experiments with n=10-11 (Panc02) or n=11-13 (KPC).

FIG. 3: Characterisation of Tumour Infiltrating Lymphocytes

C57BI/6 mice bearing KPC tumours were treated with isotype control, IL-15, CD40 agonist or the combination of the latter. Tumour were harvested 8 post treatment initiation. Single cell suspensions were acquired after enzymatic digestion for flow cytometry analysis. FIG. 3A-3I illustrate the immune cell populations indicated as fold change of absolute number of cells. Data pooled from 3 independent experiments, n=13-16/group. Two-way ANOVA with Bonferroni. * p<p≤0.05;** p≤0.01;*** p≤0.001.

FIG. 4: Characterisation of DCs in tumor and TDLN. C57BL/6j mice bearing KPC tumors were treated with isotype control, IL-15, CD40 agonist or the combination of the latter. Tumors or TDLN were harvested at day 8 post-treatment initiation. Single-cell suspensions were acquired after enzymatic digestion for flow cytometry analysis. (a, b) DCs or CD103⁺ DCs in tumors. (c, d) DCs or CD103⁺ DCs in TDLN. Data pooled from three independent experiments, n=10-16/group. One-way ANOVA with Bonferroni. *P<0.05;**P≤0.01;****P≤0.0001.

FIG. 5: Rechallenge experiments. C57BL/6j mice cured from Panc02 or KPC tumors after treatment with IL-15+CD40 agonist were re-injected with the same tumor type at the contralateral side of the abdomen. (a, b) Tumor kinetics and survival (log-rank test) of mice rechallenged with Panc02 tumor cells, n=16. (c, d) Tumor kinetics and survival of mice rechallenged with KPC tumor cells, n=9. (e, f) Flow cytometry quantification of intra-tumoral CD8⁺ Effector or Memory T cells of KPC tumor-bearing mice after 8 days following treatment (n=9). One-way ANOVA with Bonferroni.**P≤0.01;***P≤0.001;****P≤0.0001.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

When describing the compounds of the invention, the terms used are to be construed in accordance with the following definitions, unless a context dictates otherwise.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

The present invention thus relates to a combination comprising IL-15 and a CD40 agonist for use in the treatment of a pancreatic cancer.

In the context of the present invention, the term “pancreatic cancer” is meant to be disorder arising from the out of control multiplication of cells in the pancreas, i.e. a glandular organ behind the stomach. These cancerous cells have the ability to invade other parts of the body. There are a number of types of pancreatic cancer, with pancreatic adenocarcinoma accounting to about 85% of cases. One to two percent of pancreatic cancer cases are neuroendocrine tumours, which arise from the hormone-producing cells of the pancreas. Pancreatic cancer is the third most common cause of death from cancer in the United States, and most often occurs in the developed world, presumably due to the increased risk, associated with tobacco smoking, obesity and diabetes.

The inventors of the present invention have found that the combination according to the present invention exhibits enhanced anti-tumour effect resulting in profound survival increase and even complete cure of the majority of tumours. An advantage of the combination in accordance with the present invention is therefore enhanced anti-tumour activity. This activity is mediated by an enlarged infiltration of CD8 T cells and NK cells and at the same time a reduction of T regulatory cells. In the present application is provided translational preclinical data on the combination in accordance with the present invention. Moreover, the inventors have found that by using this specific combination, at least one of the components can be used at subtherapeutic dosages, thereby reducing the risk of side-effects associated with high dosages of said components.

Hence, in a particular embodiment, the present invention provides a combination comprising IL-15 and a CD40 agonist for use in the treatment of a pancreatic cancer; wherein at least one of said IL-15 and CD40 agonist are used in a subtherapeutic dose.

More in particular, the present invention provides a combination comprising IL-15 and a CD40 agonist for use in the treatment of a pancreatic cancer in a mammal such as a human being; wherein at least one of said IL-15 and CD40 agonist are used in a subtherapeutic dose.

The term “IL-15”, as used herein, refers to a cytokine that regulates the activation and proliferation of T cells and natural killer (NK) cells. Other names in the art for IL-15 include IL15 and interleukin-15. The IL-15 as used in the present invention may be used as such, or may comprise the use of recombinant forms such as rh IL15. Alternatively, it may also comprise the use of IL-15 agonists and superagonists, such as for example RLI-15, IL15 SA or N-803. N-803, formerly ALT-803, is an IL-15 superagonist mutant and dimeric IL-15RαSushi-Fc fusion protein complex that enhances CD8⁺ T and NK cell expansion and function and exhibits anti-tumor efficacy in preclinical models. IL15 SA combines IL-15 and IL15Ra-Fc. RLI-15 is a fusion protein consisting of the NH2-terminal (amino acids 1-77, sushi+) cytokine-binding domain of IL-15Ra coupled to IL-15 via a 20-amino acid flexible linker. This fusion protein, referred to as protein receptor-linker-IL-15 (RLI) acts as an IL-15 superagonists that has an increased serum half-life and biological activity similar to complexed IL-15/IL-15Rα-Fc.

The term “CD40 agonist”, as used herein, refers to a molecule which specifically binds to the subject's CD40 molecule and increases or enhances or induces one or more CD40 activities when it comes in contact with a cell, tissue or organism of the subject expressing CD40.

As used herein, “CD40” refers to a cell surface glycoprotein that is a member of the tumor necrosis factor receptor family. Other names in the art for CD40 include: TNFRSF5, p50, CDW40 and Bp50.

In accordance with an embodiment of the present invention, said CD40 agonist is an antibody or antigen binding fragment thereof such as selected from: Selicrelumab (also known as RG 7876), APX005M, ChiLob7/4, ADC-1013, SEA-CD40, CDX-1140. In accordance with an embodiment of the present invention, said CD40 agonist may also be selected from: CD40L, trimers of CD40L, HERA-CD40L.

The term “antibody” as used herein can include whole antibodies, F(ab′)2 fragment, diabody, triabody, tetrabody, bispecific antibody, monomeric antibodies and any antigen binding fragment (i.e., “antigen-binding portion”) or single-chain variable region fragment (scFv), or disulfide-stabilized variable region fragment (dsFv) thereof. Whole antibodies are glycoproteins comprising at least two heavy (H) chains and two light (F) chains inter-connected by disulfide bonds. Each heavy chain is comprised of a heavy chain variable region (abbreviated herein as VH) and a heavy chain constant region.

In all different embodiments of the present invention, the pharmaceutical combination according to the invention can be administered in a therapeutic or subtherapeutic daily dose to a subject, in particular a human subject. In yet another embodiment, at least one of the components of the pharmaceutical combination of the present invention is administered in a subtherapeutic dose to a subject. In another embodiment, at least one of the components of the pharmaceutical combination according to this invention is administered in a therapeutic dose. In still another embodiment, one of the components of the pharmaceutical combination is administered in a subtherapeutic dose, whereas the other component is administered in a therapeutic dose. In another embodiment, all components of the pharmaceutical combination of the present invention are administered in a subtherapeutic dose to the subject.

In the context of the present invention, a subtherapeutic dose of a therapeutic compound is meant to be a dose which is lower than the usual/typical dose of said compound required to obtain a therapeutic effect. Accordingly, in a preferred embodiment, at least one of the components is administered at a dose that is lower than its dose required to obtain a therapeutic effect, when administered alone.

The term “subtherapeutic amount” of a compound means an amount that would be below an accepted therapeutically effective amount. A subtherapeutic amount can be defined as an amount less than the FDA-approved dosage or dosages for a particular disease. Alternatively, taking into account that many drugs are used off-label, a subtherapeutic amount can be defined as an amount less than that typically prescribed by physicians for a particular disease. A sub-therapeutic amount may also take into account such factors as body mass, sex, age, renal or hepatic impairment, and other parameters which may affect the efficaciousness of a given amount of a particular drug. In certain embodiments, a subtherapeutic amount may be 85% of a therapeutically effective amount. In further embodiments, a subtherapeutic amount may be 70% of a therapeutically effective amount. In further embodiments, a subtherapeutic amount may be 60% of a therapeutically effective amount. In further embodiments, a subtherapeutic amount may be 50% of a therapeutically effective amount. In further embodiments, a subtherapeutic amount may be 40% of a therapeutically effective amount. In further embodiments, a subtherapeutic amount may be 30% of a therapeutically effective amount. In further embodiments, it may be even less.

In other words, a subtherapeutic dose is an amount of a compound/component, which when administered to a patient, is not in itself sufficiently effective in the treatment of the claimed disorders. Yet, the combination as claimed herein was found to be sufficiently effective in the treatment of the claimed disorders, even if one or more of the compounds/components were administered at doses which are not sufficiently effective in the treatment of the claimed disorders.

Where in the context of the present invention, reference is made to a particular dose, this is meant to be the dose to be administered on a given day, even where not explicitly stated so in the current application. For example, where it is stated that the CD40 agonist is administered at a dose of about 20 μg per kg body weight, this is meant to be understood as being administered at a dose of about 20 μg per kg body weight per day. This does not mean that during the treatment regime the dose is given every day of the treatment regime, but rather that on each day of administration of the dose, the given dose per kg body weight is as stated herein.

In a preferred embodiment, in the pharmaceutical combination, the CD40 agonist is administered/used in a dose of from about 20 to about 800 μg per kg body weight, preferably from about 30 to about 600 μg per kg body weight, most preferably from about 40 to about 300 μg per kg body weight. Therapeutic doses currently used in clinical trial typically exceed 300 μg per kg body weight. Depending on the type of CD40 agonist, the subtherapeutic dose may vary. For example, clinically used therapeutic doses for Selicrelumab, amount to about 200 μg whilst for another CD40 agonist, APX005M, the therapeutic dose is 300 μg. Therefore, in a preferred embodiment, the CD40 agonist is administered at a subtherapeutic dose of less than 300 μg per kg body weight; such as less than 250 μg per kg body weight, less than 200 μg per kg body weight; less then 150 μg per kg body weight; less than 100 μg per kg body weight.

In another preferred embodiment, in the pharmaceutical combination, the IL-15 or agonist thereof is administered/used in a dose of from about 0.1 to about 50 μg per kg body weight, preferably from about 0.1 to 20 μg per kg body weight, most preferably from about 0.1 to about 0.3 μg per kg body weight. The dose of administration of IL-15 changes in relation to the method of administration. In particular, when IL-15 is administered intravenously, IL-15 is administered typically at a dose of approximately 0.3 μg per kg body weight in a bolus IV injection, whilst when IL-15 is administered subcutaneously, IL-15 is administered typically at a dose up to 2 μg per kg body weight.

Hence in a particular embodiment, said IL-15 is administered intravenously at a dose of less than 0.3 μg per kg body weight, such as about 0.2 or about 0.1 μg per kg body weight. In particular where IL-15 is administered via a bolus IV injection, the dose may be less than 0.3 μg per kg body weight, such as about 0.2 or about 0.1 μg per kg body weight. Alternatively, where IL15 is administered via a continuous IV drip system, the dose may be about or below 2 μg per kg body weight, such as about or below 1.5, about or below 1.0, about or below 0.5 μg per kg body weight. Alternatively, said IL-15 is administered subcutaneously or intradermally at a dose of less than 2 μg per kg body weight; such as about 1.5 or about 1 μg per kg body weight.

Therapeutic doses currently used in bolus IV injections in clinical trial typically exceed 0.3 μg per kg body weight. Therefore, in a preferred embodiment, the IL-15 or agonist thereof is administered at a subtherapeutic dose of less than 0.3 μg per kg body weight; such as less than 0.2 μg per kg body weight, less than 0.1 μg per kg body weight.

Hence, in a specific embodiment the present invention provides a combination as defined herein, wherein at least one of the following applies:
said CD40 agonist is used at a subtherapeutic dose of less than 300 μg per kg body weight;
said IL-15 is administered intravenously at a subtherapeutic dose of less than 0.3 μg per kg body weight;
said IL-15 is administered via a bolus injection intravenously at a dose of less than 0.3 μg per kg body weight;
said IL-15 is administered via a continuous drip system intravenously at a dose of about or below 2 μg per kg body weight; or
said IL-15 is administered intradermally or subcutaneously at a subtherapeutic dose of less than 2 μg per kg body weight.

The inventors have surprisingly found that a striking dose reduction of CD40 agonist was possible by adding IL-15. An advantage of this embodiment is that adverse effects deriving from the use of the CD40 agonist can be reduced. Moreover, given this synergistic effect of both molecules, in a particular embodiment, both components may even be used at a subtherapeutic doses.

By “combination comprising IL-15 and a CD40 agonist”, as used herein, it is referred to any form comprising the said IL-15 and said CD40 agonist which can be administered to a subject. As used herein “a combination” refers to any association between two or more items. The association can be spatial or refer to the use of the two or more items for a common purpose. Hence, in the context of the present invention, the combination as provided herein may be a single composition comprising both components. However, a combination in the context of the invention, also refers to the use of 2 separate compositions, each comprising one of the components, which are nevertheless used in association with each other in the claimed treatment. Said association, may include the simultaneous administration of both compositions to the patient, or the separate administration in such a manner that both components are still able to cooperate in the treatment of the intended disorders.

In another particular embodiment of the present invention, the combination is in the form of a pharmaceutical composition. The pharmaceutical compositions in accordance with the present invention can be for use in human or veterinary medicine.

As used herein, a “composition”, refers to any mixture of two or more products or compounds (e.g. agents, modulators, regulators, etc.). It can be a solution, a suspension, liquid, powder or a paste, aqueous or non-aqueous formulations or any combination thereof. In the context of the present invention, the compositions are preferably pharmaceutical compositions, comprising one or more pharmaceutically excipients, carriers, diluents, . . . .

In the context of the present application, the terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptoms but has not yet been diagnosed as having it; (b) inhibiting the disease symptoms, i.e. arresting its development; or (c) relieving the disease symptom, i.e. causing regression of the disease or symptom.

It has to be understood that different methods of administration are suitable to administer the combination in accordance with the present invention. Administration of the combination according to the present invention can be performed using standard routes of administration. Non-limiting embodiments include parenteral administration, such as intradermal, intramuscular, subcutaneous, transcutaneous, or mucosal administration, e.g. intranasal, oral, and the like. In one embodiment in accordance with the present invention, the combination comprising said IL-15 and said CD40 agonist are administered intravenously or subcutaneously. The intravenous administration can be done via a bolus injection or via a continuous IV drip system. The route of administration may determine the therapeutic efficacy, as it was found that subcutaneous administration gives better results than IV bolus injection. In yet a further embodiment, the pancreatic cancer is selected from: pancreatic ductal adenocarcinoma, pancreatic neuroendocrine tumours.

In a further aspect, the present invention provides a method for the treatment of a pancreatic cancer in a subject in need thereof, said method comprising administering to said subject, a combination comprising IL-15 and a CD40 agonist; wherein at least one of said IL-15 and CD40 agonist are used in a subtherapeutic dose.

### Examples

Material and Methods

Animals

Female C57BL/6J mice, age 6-8 weeks, were obtained from Jackson Laboratories. All mice were maintained at the Animal Core Facility at the University of Antwerp. All animal procedures were conducted in accordance with, and approval of, the Animal Ethics Committee of the University of Antwerp under registration number 2016-30. All mice were housed in filter-top cages enriched with houses and nesting material. Mice were checked on a daily base to inspect health and wellbeing. Mice were given a 7 days adaptation period upon arrival before being included in experiments to reduce stress levels.

Models and the Experimental Design

Two different mouse pancreatic ductal adenocarcinoma cell lines were used. Panc02 is a chemically induced cell line while the KPC cell is derived from on an orthotopic KPC tumour bearing the KRAS and p53 mutation. Both cell lines were cultured in DMEM cell culture medium supplemented with 10% FBS and 10 mM L-Glutamine. Cell lines were maintained at 37° C. and 5% CO2. All cell lines were tested on a routine base for mycoplasma contamination. All cell lines were not passaged more than 10 times between freeze and thawing and were only used in experiments between passage two and six.

Tumour Kinetics and Survival

Prior to injection, Panc02 and KPC cell were harvested using Tryple, washed trice with sterile PBS and put on a 70 μm cell strainer to assure single cell suspension without any contaminants. Next, mice were injected subcutaneously with either 0.5.10⁶ Panc02 or KPC cells per suspended in 100 μL sterile PBS at the left abdominal flank. When tumour reached an average size of 20-30 mm², mice were randomised based on tumour size and divided over four different treatment groups (=day 0): 1. Isotype control; 2. IL-15; 3. αCD40; 4. IL-15+αCD40. Mice were given i.p. 2.5 μg IL-15 (NCI) at days 0-3, 6-10 and 13-14. A mouse agonistic CD40 monoclonal antibody (FGK-45) or corresponding isotype (2A3) was administered i.p. at days 0, 3, 7, 10 and 14 at a dosage of 12.5 μg per mouse for Panc02 or 200 μg (day 0) and 100 μg (days 3, 7, 10 and 14) for KPC tumours.

Tumour size was measured twice a week using a digital calliper (Chicago Brand). Tumour area was calculated using the formula length×width. Mice were sacrificed when a tumour size of 150 mm² was reached or were stated as long-term survivor when they reached day 100 without reaching this endpoint.

Functional Depletion Experiments

For investigation of the role of different immune cell types, functional depletion experiments were carried out. Mice were given Panc02 or KPC tumour as described above. CD4 and CD8 T cells were depleted using 200 μg of monoclonal antibodies and NK cells were depleted using 25 μl of anti-asialo-GM1 per mice. Mice were randomised in six different treatment groups: 1. Isotype control; 2. IL-15+αCD40; 3. IL-15+αCD40+αCD4; IL-15+αCD40+αCD8; IL-15+αCD40+anti-Asialo-GM1; IL-15+αCD40+αCD8+anti-Asialo-GM1. Depletion antibodies or anti-asioalo-GM1 were given i.p. at days−1, 0, 3, 6, 10 and 14. Tumour kinetics and survival were measured as described above.

Re-Challenge Experiments

To investigate induction of immune memory, re-challenge experiments were performed. Here, mice who were completely tumour-free 100 days post start of treatment, were re-injected s.c. with either Panc02 or KPC cells at the contralateral flank of the primary tumour injection side. Tumour growth and survival were measured as described above.

Characterisation of TIL

To characterise tumour infiltrating lymphocytes, multicolour flow cytometry experiments were performed on both spleen and tumour from different treatment groups. Here, mice bearing Panc02 or KPC tumour were randomised and treated as described above. At day 8, mice were sacrificed and both spleen and tumour were removed during necropsy and weighed. Next, tumours were minced using scalpels followed by enzymatic digestion with digestion medium (RPMI 1640+10% FBS+10 mM L-glutamine+Collagenase D+DNAse-I+Liberase) for 30 min at 37° C. and 5% CO₂ at a cell rocker. After digestion, all samples were washed with wash buffer (PBS+2% FBS+1 mM EDTA) and put through a 70 μm cell strainer to obtain single cell suspension. Spleens were dissociated mechanically, washed with FACS buffer and put through a 40 μm cell strainer to obtain single cell suspension.

Both single cell suspensions of spleen and tumour were stained with three different multicolour antibody panels. Panel 1 consists of CD107a-BV421, CD8-FITC, CD3-PE, CD4-PercP-Cy5.5, CD69-Pe-Cy7, NK1.1-APC, panel 2 of CD8-BV421, CD25-BV786, CD4-FITC, CD3-PE, CD44-PerCP-Cy5.5, CD62L-Pe-Cy7, FoxP3-APC, panel 3 of CD8-BV421, CD103-BV786, Ly6G-FITC, CD11b-PE, F4/80-Pe-TexasRed, Ly6C-PerCP-Cy5.5, MHC-II-PE-Cy7, CD11c-APC. In all three panels, Live-dead Aqua was used as a viability staining and CD45.2-APC-Cy7 was used to gate out leucocytes and not tumour cells. All cell suspensions were blocked using 2.4G2 prior to antibody staining. All samples were analysed using a FACS Aria II flow cytometer.

Statistics

Statistical differences in tumour kinetics between different treatment groups in different experiments were determined using a two-way ANOVA with Bonferroni post-hoc analysis. Differences in survival were analysed using a Log-rank test. To assess a difference between the amount of TIL in tumour by flow cytometry, a two-ANOVA with Bonferroni posthoc analysis was applies. Differences were considered to be significantly different if p<0.5. Graphs were made using GraphPad v8.0 software. Flow cytometry analysis was performed using FlowJo v10.6.3 All statistical analyses were carried out in SPSS v26.

Results

The Combination of IL-15 with a CD40 Agonist Results in Decreased Tumour Volume and Increased Survival

We sought to investigate whether combined treatment of IL-15 and a CD40 agonist can lead to augmented anti-tumour responses in pancreatic ductal adenocarcinoma. To investigate this, C57BL/6 mice bearing either Panc02 or KPC tumours were treated with IL-15 and/or a CD40 agonist intraperitoneally when tumours reached a size of 25-35 mm² (FIG. 1A). We show here that the combination of these agents results in decreased tumour volumes and increased survival in both mouse models.

For the first model, Panc02, we observed a significant decrease in tumour volume when mice were treated with single agents. Treatment with the combination regimen resulted in significant reduction of tumour volume, even when compared to both single agent treatments, and an increased survival with 16 out of 17 mice becoming completely tumour free (FIG. 1B, 1D, 1F).

In the second model, KPC, we observed very similar results: reduced tumour volumes and increased survival were demonstrated with the combination being significantly better than control or either single agent. 7 out of 11 mice became again completely tumour free (FIG. 1C, 1E, 1G).

Remarkably, while we observed similar responses between the Panc02 and KPC model, the required CD40 agonist dose in the Panc02 model (12.5 μg/dose; Human Equivalent Dose is 51 μg/kg) is 8 times lower than in the KPC model (200 μg and 100 μg/dose; Human Equivalent Dose is 813 μg/kg and 406 μg/kg respectively). Therefore, depending on the tumour type, combination of the two agents might result in a profound dose reduction of the CD40 agonist. Summarized, we show here that the combination of IL-15 and CD40 agonist has profound anti-tumour activities where combining both agents leads to additive effects.

CD8⁺ T Cells, NK Cells are Responsible for the Main Anti-Tumour Effector Cells

To gain more insight into the immune cells responsible for the observed anti-tumour responses, we depleted several immune cell populations in tumour-bearing mice using specific depletion antibodies. Next, we observed the effect this depletion had on both tumour kinetics and survival (FIG. 2A, 2E). Upon depletion of CD4⁺ T cells, we observed in both PDAC models that there was no significant difference in survival between the depleted and non-depleted group, indicating that the CD4⁺ T cells do not play a significant role in the anti-tumour response elicited by our combination treatment (FIG. 2B, 2F). However, when CD8⁺ T were depleted, the anti-tumour effect of the combination treatment was significantly reduced in both tumour models. There was a significant increase in tumour growth and a reduced survival (FIG. 2C, 2G). When looking at the role of NK cells, the observed results show a mixed response: in the Panc02 model, we could clearly observe a significant decrease in tumour survival although not in tumour growth, while for the KPC model a decrease in survival was observed although not statistically significant (FIG. 2D, 2H). In conclusion, these experiments show that CD8⁺ T cells are mainly responsible for the observed anti-tumour effects with a trend towards involvement of NK cells as well, whereas CD4⁺ T cells do not play a significant role.

The Combination of IL-15 with a CD40 Agonist Results in Increased Amounts of Anti-Tumour Immune Cells in the Tumour

To further explore the anti-cancer effects of the combination regimen, multi-colour flow cytometry to chart the tumour infiltrating lymphocytes was performed. We observed interesting differences between the different arms of this combination therapy. The immune cell populations treated were: Leucocytes (FIG. 3A), NK cells (FIG. 3B), NK T cells (FIG. 3C), T cells (FIG. 3D), CD4 T cells (FIG. 3E), CD8 T cells (FIG. 3F), Tregs (FIG. 3G), dendritic cells (DC) (FIG. 3H), neutrophils (FIG. 3I). The first observation is that the anti-tumour effect is not due to a higher amount of infiltrating lymphocytes as such but rather caused by significant differences among the present immune cell subsets (FIG. 3A). First, the amount of infiltrating NK and NK T cells is significantly higher in the combination group compared to the isotype and CD40 agonist group. IL-15 alone shows an increased amount of both cell types though not statistically significant (FIG. 3B, 3C). Regarding the T cells, we do observe an increased amount of total T cells in the combination regimen (FIG. 3D). When zooming in on this T cell compartment, it is clear that this increased amount of T cells can be attributed to the CD8+ T cells which are significantly higher in the combination when compared to all other arms while there is no difference in infiltrating CD4 T cells (FIG. 3E, 3F). Interestingly, we also observed a significant decrease in Tregs present in the tumour when CD40 agonist was part of the treatment (FIG. 3G). Next, also the amount of intratumoural DC is increased in the combination group, indicating a better priming of CD8 T cells and NK cells (FIG. 3H). Finally, no significant differences in the amount of present neutrophils were observed (FIG. 3I). Putting these observations on NK cells, CD8+ T cells, DC and Tregs together, it makes sense that the combination treatment has a more profound anti-tumour effect as it causes a better priming of the anti-cancer immune cells (DC), followed by a significant increase in anti-tumour immune cells (NK, NK T and CD8+ T cells) combined with a decrease of immunosuppressive cells, being the Tregs, also evidenced by the increased CD8/Treg ratio.

Combination Therapy Results in Increased Amounts of CD103⁺ Cross-Presenting DCs

Dendritic cells are known to play critical roles in antigen processing and presentation and are key players in the activation of both NK and T cells. We further explored their presence in the KPC tumor model. Here, we observed a significant increase in number of DCs in the tumor, only in the combination therapy group (FIG. 4a). Furthermore, the amount of CD103⁺ DCs, the subtype responsible for cross-presentation, was determined. Here, IL-15 caused a significant increase in the number of CD103⁺ DCs in the tumor while this was significantly lower in the groups following treatment with CD40 agonist (FIG. 4b). To further investigate how the DCs behaved, we analysed the presence of these cells in the tumor-draining lymph nodes (TDLN) and observed a 3-fold increase in number of DCs when CD40 agonist was administered (FIG. 4c). The frequency of CD103⁺ cross-presenting DCs increased twofold under these conditions (FIG. 4d), suggesting that these DCs captured antigens at the tumor site and migrated to the TDLN to activate NK and T cells. Furthermore, gene signatures showed a higher expression of CD80, CD83 and CD86 when the combination therapy was administered, indicating that likely antigen-presenting cells like DCs are activated and mature. The increase in mRNA encoding expression of CXCR3, CXCL9 and CXCL10 (not CXCL11) as chemotactic chemokines suggests their involvement in the trafficking of anti-tumor immune cells to the tumor site (data not shown).

Induction of Immune Memory

One of the goals of immunotherapy is the induction of strong immunological memory to prevent future relapse. We observed in our study an increased number of effector and central memory CD8⁺ T cells in KPC tumors when treated with the combination regimen, compared to isotype control or single arm treatments (FIGS. 5a and b). To investigate whether functional immune memory was induced, tumor-free mice from the combination treatment group were rechallenged with the same tumor cell type as they were originally inoculated with. Here, we observed clear induction of immune memory in both PDAC models with 14 out of 16 mice becoming tumor free for the Panc02 tumor model and all mice becoming tumor free for the KPC tumor model compared to naive control mice (FIG. 5c-f).

1. A combination comprising IL-15 and a CD40 agonist for use in the treatment of a pancreatic cancer in a mammal; wherein at least one of said IL-15 and CD40 agonist are used in a subtherapeutic dose.

2. The combination of claim 1, wherein said subtherapeutic dose of said IL-15 and CD40 agonist is a dose which is lower than the dose of said IL-15 and CD40 agonist required to obtain a therapeutic effect in said mammal, when administered alone.

3. The combination according to anyone of claim 1 or 2, wherein said CD40 agonist is used at a dose of from about 20 to about 800 μg per kg body weight, preferably from about 30 to about 600 μg per kg body weight, most preferably from about 40 to about 300 μg per kg body weight.

4. The combination according to anyone of claims 1 to 3, wherein said IL-15 is used at a dose of from about 0.1 to about 50 μg per kg body weight, preferably from about 0.1 to 20 μg per kg body weight, most preferably from about 0.1 to about 2 μg per kg body weight.

5. The combination according to anyone of claims 1 to 4, wherein said IL-15 is administered intravenously via an IV bolus injection at a dose of less than 0.3 μg per kg body weight; or via a continuous IV drip system at a dose of about or below 2 μg per kg body weight.

6. The combination according to anyone of claims 1 to 5, wherein said IL-15 is administered subcutaneously or intradermally at a dose of less than 2 μg per kg body weight.

7. The combination according to anyone of claims 1 to 6; wherein at least one of the following applies:
said CD40 agonist is used at a dose of less than 300 μg per kg body weight;
said IL-15 is administered via a bolus injection intravenously at a dose of less than 0.3 μg per kg body weight;
said IL-15 is administered via a continuous drip system intravenously at a dose of about or below 2 μg per kg body weight; or
said IL-15 is administered subcutaneously or intradermally at a dose of less than 2 μg per kg body weight.

8. The combination according to anyone of claims 1 to 7, wherein the combination is in the form of a pharmaceutical composition.

9. The combination according to anyone of claims 1 to 8, wherein said CD40 agonist is a CD40 antibody or antigen binding fragment thereof such as selected from: Selicrelumab, APX005M, ChiLob7/4, ADC-1013, SEA-CD40, CDX-1140.

10. The combination according to anyone of claims 1 to 9, wherein said CD40 agonist is selected from: CD40L, trimers of CD40L, HERA-CD40L.

11. The combination according to anyone of claims 1 to 10, wherein said IL-15 and said CD40 agonist are administered simultaneously.

12. The combination according to anyone of claims 1 to 11, wherein said IL-15 and said CD40 agonist are administered intravenously, intradermally or subcutaneously.

13. The combination according to anyone of claims 1 to 12, wherein the pancreatic cancer is selected from: pancreatic ductal adenocarcinoma, pancreatic neuroendocrine tumours.

14. A method for the treatment of a pancreatic cancer in a subject in need thereof, said method comprising administering to said subject, a combination comprising IL-15 and a CD40 agonist; wherein at least one of said IL-15 and CD40 agonist are used in a subtherapeutic dose.
